(19) United States
(12) Patent Application Publication
(10) Pub. No.: US 2018/0303656 A1
(43) Pub. Date: Oct. 25, 2018

(54) Mouthpiece for treating sleep apnea syndrome patients

(71) Applicant: Koji IKEJIRI, Toyonaka (JP)

(72) Inventor: Ryoji IKEJIRI, Toyonaka (JP)

(73) Assignee: KOJI IKEJIRI

(21) Appl. No.: 15/770,272

(22) PCT Filed: Oct. 11, 2016
(86) PCT No.: PCT/JP2016/080117

§ 371 Date: Apr. 23, 2018

(30) Foreign Application Priority Data
Oct. 30, 2015 (JP) JP2015-213590

(51) Int. Cl. A61F 5/56; A61C 7/08

(52) U.S. Cl. CPC A61F 5/566; A63B 71/085; A61F 2005/563; A61C 7/08

(57) ABSTRACT

A novel type of upper jaw- and lower jaw-mouthpieces are provided. The mouthpieces are two separate upper jaw- and lower jaw-mouthpieces (1, 2). The upper jaw- and lower jaw-mouthpieces (1, 2) are bonded and fixed with at least one adhesive piece (3, 31 to 36), and the adhesive piece (3, 31 to 36) allows the upper jaw- and lower jaw-mouthpieces (1, 2) to be movable within a limited range. The limited range is, for example, movement of the upper jaw- and lower jaw-mouthpieces (1, 2) upward, downward, forward, rearward, leftward, and rightward within a range of 1 to 8 mm.

### TECHNICAL FIELD

The present invention mainly relates to a therapeutic mouthpiece for sleep apnea syndrome and snoring associated therewith, but also can be used as a mouthpiece for correction of the irregular teeth and the mandibular position, and other general uses, of course.

### BACKGROUND ART

Sleep apnea syndrome (SAS) is a kind of sleep disorder, and is defined as a condition in which an apnea lasting 10 seconds or more during sleep occurs 30 times or more each night, or an average of 5 times or more per hour of sleep.

Since breathing stops during sleep, many patients cannot have sound sleep, experience excessive sleepiness during daytime, and complain of conditions such as accumulated fatigue, headaches, and reduction in concentration and memory. Such excessive sleepiness and lack of concentration during daytime can cause traffic accidents and industrial accidents, resulting in social problems. In addition, there is a concern that a reduced supply of oxygen during sleep may induce hypoxemia, and increase the risk of cardiovascular diseases such as hypertension, cardiac insufficiency cerebral apoplexy, angina pectoris, and acute myocardial infarction. Furthermore, patients with sleep apnea syndrome are said to have a high frequency of suffering from a complication of insulin-non-dependent diabetes, and the frequency of complication is also said to increase as the sleep apnea becomes more severe.

The sleep apnea syndrome is classified into three types, namely obstructive sleep apnea syndrome, central sleep apnea syndrome, and mixed sleep apnea syndrome, with the obstructive sleep apnea syndrome being most common. The obstructive sleep apnea syndrome develops as a result of the tongue root portion or the soft palate being lowered by muscle relaxation during sleep, thus obstructing the air passage. Obesity, an enlarged tonsil, a small lower jaw, and the like make the obstructive sleep apnea syndrome more likely to develop.

As a method of treatment of the obstructive sleep apnea syndrome, it is common to wear a mouthpiece or use continuous positive airway pressure (CPAP).

As a mouthpiece of this kind, the inventions described in JP 2010-29615 and JP 2005-312853 have been known.

JP 2010-29615 is characterized in that an upper jaw-mouthpiece and a lower jaw-mouthpiece are integrally molded, and suction portions, internal paths, and adsorption ports are formed in one or both of the upper jaw- and lower jaw-mouthpieces.

JP 2005-312853 is characterized in that an upper jaw-mouthpiece and a lower jaw-mouthpiece are separate, and a lower jaw slip prevention plate extending from the outer aides of the front teeth portion of the lower jaw to the outer sides of the front teeth portion of the upper jaw-mouthpiece is attached to the lower jaw-mouthpiece.

According to the descriptions of the two inventions, the wearing of the mouthpieces during sleep prevents the tongue root portion from drooping rearward during sleep to obstruct the air passage, and expands the air passage, thus alleviating the obstructive sleep apnea and snoring.

### PRIOR ART DOCUMENTS

### Patent Documents

Patent Document 1: JP 2010-29615

Patent Document 2: JP 2005-312853

### DISCLOSURE OF INVENTION

### Problem to be Solved by the Invention

In the former invention, i.e., JP 2010-29615, the upper law- and lower jaw-mouthpieces are integrated with each other. Since the physiological activity of the lower jaw is forcibly restrained, it is very difficult, particularly, for a patient with temporomandibular, to cause the lower jaw to protrude forward and to be fixed and held for a long period of time. In addition, the outer sides of the upper and lower root rows are surrounded by a horseshoe-shaped well. Accordingly, the structure makes mouth breathing difficult, and thus cannot cope with coughing and sneezing during sleep, making the wearing of the mouthpieces for a long period unrealistic.

In the latter invention, i.e., JP 2005-312853, the upper jaw- and lower jaw-mouthpieces are separate, and, therefore, the problem of the former invention has ben solved. However, the lower jaw-mouthpiece is disposed forward of the upper jaw-mouthpiece, and thus forces the patient to have a posture that is more unnatural than that forced by the integral mouthpiece. Moreover, the relative movement between the upper jaw- and lower jaw-mouthpieces is free, and therefore, the problem of whether the unnatural posture can be maintained during sleep remains.

It is an object of the present invention to provide a novel type of an upper jaw- and lower jaw-mouthpieces.

### Means for Solving Problem

A mouthpiece according to the present invention includes two separate upper jaw- and lower jaw-mouthpieces, wherein the upper jaw- and lower jaw-mouthpieces are bonded and fixed with at least one adhesive piece, and the adhesive piece allows the upper jaw- and lower jaw-mouthpieces to be movable within a limited range.

Preferably, the limited range allows the upper jaw- and lower jaw-mouthpieces to move in all directions, for example, upward, downward, forward, rearward, leftward and rightward within a range of 1 to 8 mm, preferably 2 to 6 mm.

Preferably, a raw material of the adhesive piece is one of an inorganic fiber such as a glass fiber or a carbon fiber, and an organic fiber such as an aramid fiber or a polyethylene fiber.

Preferably, the adhesive piece is a twilled fabric.

Preferably, a quick cure resin is used as an adhesive material used for attaching the adhesive piece to the upper jaw- and lower jaw-mouthpieces.

In another modification, the adhesive piece is configured to be attachable and removable to and from the upper jaw- and lower jaw-mouthpieces, without using the adhesive material.

To allow the adhesive piece to be attachable and removable to and from the upper jaw- and lower jaw-mouthpieces, a female or male snap member can be disposed on the adhesive piece and the upper jaw- and lower jaw-mouthpieces, for example.

The present invention also can be regarded as an adhesive piece for use in bonding and fixing upper jaw- and lower jaw-mouthpieces so as to be movable relative to each other within a limited range.

Preferably, the limited range allows the upper jaw- and lower jaw-mouthpieces to move at least forward, rearward, leftward, and rightward within a range of 1 to 8 mm. The adhesive piece is preferably one of an inorganic fiber such as a glass fiber and a carbon fiber or an organic fiber such as an aramid fiber or a polyethylene fiber, and is preferably a twilled fabric. Furthermore, the adhesive piece preferably has been subjected to an end treatment such as formation of a lug portion so as to prevent end thereof from becoming loose, and preferably has been subjected to a pretreatment such as impregnation with a thermosetting resin, a chemical polymerization resin, a photopolymerization resin, or the like.

### Effects of the Invention

According to the present invention, the upper jaw- and lower jaw-mouthpieces are bonded and fixed with at least one adhesive piece, and the adhesive piece allows the upper jaw- and lower jaw-mouthpieces to move within a limited range. That is, the upper and lower mouthpieces are in an intermediate state in which they are not completely freely separated, or are not completely restrained and fixed.

In this intermediate state, a patient with sleep apnea syndrome will not undergo obstruction of the air passage as a result of the tongue root portion drooping rearward during sleep. Moreover, the patient can move the lower jaw a limited range even during sleep, and thus experiences a significantly reduced distress as compared with when the mouthpieces are completely fixed.

Hereinafter, embodiments of the present invention will be described with reference to the accompanying drawings.

### BRIEF DESCRIPTION OF DRAWINGS

FIG. 1 is a side view of a state in which a mouthpiece according to the present invention is worn.

FIG. 2(a) is a front view of the mouthpiece of the present invention, FIG. 2(b) is plan view thereof FIG. 2(c) is a side view thereof, and FIG. 2(d) is a cross-sectional end view taken along the arrows in FIG. 2(a).

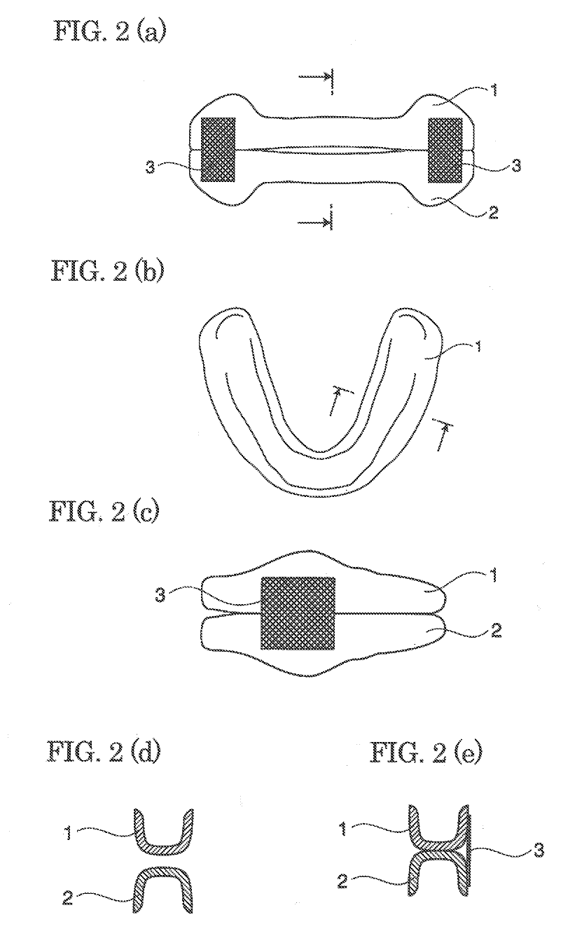

FIGS. 3(a) to (d) are side views showing movements of the mouthpiece according to the present invention, and FIG. 3(e) is a front view thereof.

FIG. 4(a) is a cross-sectional view of a human face showing a state of the air passage when the mouthpiece according to the present invention is not worn. FIG. 4(b) is a cross-sectional view of a human face showing a state of the air passage in a state in which the mouthpiece is worn.

FIGS. 5(a) to 5(e) are modifications of the mouthpiece according to the present invention.

FIG. 6 shows still another modification of the mouthpiece according to the present invention, wherein FIG. 6(a) shows a side view, FIG. 6(b) is a cross-sectional view taken along the arrow, and FIG. 6(c) is a partial enlarged view.

### MODES FOR CARRYING OUT THE INVENTION

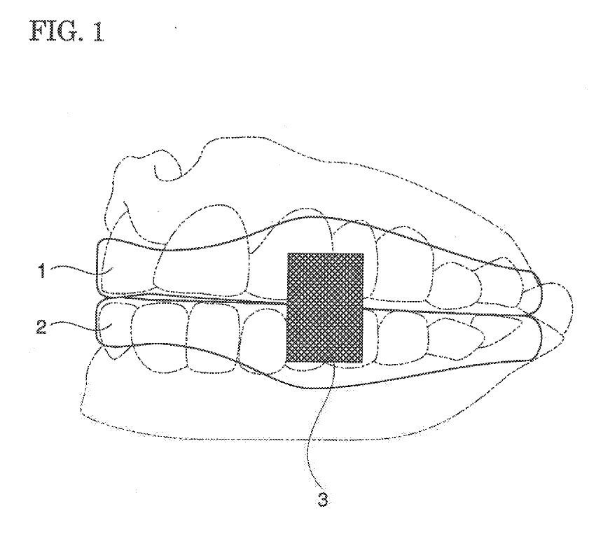

As shown in FIG. 1, a mouthpiece according to the present invention includes two separate upper jaw- and lower jaw-mouthpieces 1 and 2 (hereinafter also abbreviated as “upper and lower mouthpieces”). As is well known in the dentistry industry, each mouthpiece is formed of a thermoplastic resin, and specifically is formed by heating and pressurizing a thermoplastic resin sheet.

The raw material of the thermoplastic resin sheet is not limited, as long as it is a resin that can be hot-formed. Examples of the raw material that can be used include ethylene-vinyl acetate copolymers (EVA), polyolefins (e.g., polyethylene, polypropylene, polybutadiene, and copolymers of monomers constituting these polymers), polyvinyl acetate (PVA), and polyurethane. Among these, ethylene-vinyl acetate copolymers (EVA), polyolefins, and polyurethane are preferable.

A feature of the present invention lies in that the upper and lower mouthpieces 1 and 2 are fixed with at least one adhesive piece 3, and the adhesive piece 3 allows the upper and lower mouthpieces 1 and 2 to move within a limited range.

The adhesive piece 3 is used in an oral cavity, and is used constantly during sleep over a long period. Therefore, it is essential that the raw material is durable, harmless to the human body, and moreover have affinity for an adhesive. In addition, the adhesive piece 3 is required to allow the upper and lower mouthpieces 1 and 2 to be movable within a limited range.

Examples of the raw materials that are considered to be usable at present include inorganic fibers such as a glass fiber and a carbon fiber and organic fibers such as an aramid fiber and a polyethylene fiber. However, the raw material is not particularly limited thereto, and two or more of these reinforcement fibers may be used in combination.

The adhesive piece is used as a woven piece or a knitted piece that is sized to be attachable to the upper and lower mouthpieces 1 and 2. In general, the adhesive piece has a square shape, but may be another shape, including, for example, a cross shape, an X-shape, a diamond shape, a trapezoidal shape, a circular shape, and an oval shape. Examples of the weaving method include plain weaving, diagonal weaving (twill weaving), satin weaving, modified plane weaving, modified twill weaving, modified satin weaving, fancy weaving, brocade, double texture, multiple texture, warp pile weaving, weft pile weaving, and interweaving. Examples of the knitting include circular knitting, weft knitting, warp knitting, and pile knitting, and there are plain stitch, circular rib (rib stitch, cardigan stitch), smooth knitting (double knitting), rib knitting, purl knitting, Denbigh texture, cord texture, atlas texture, chain stich structure, insertion texture, and so forth. Among these, a diagonal woven fabric is preferable because of ease of movement of the adhesive piece.

Although there is also no limitation on the position to which the adhesive piece 3 is attached and the number of the positions, it is preferable that the adhesive piece is attached to side portions at 2 to 4 locations. Besides, the adhesive piece 3 may be attached to the position of the front teeth.

As the adhesive material used for attaching the adhesive piece 3 to the mouthpieces 1 and 2, a quick cure resin is preferable because it is safe and durable. As the quick cure resin, a quick cure resin composed of a polymerizable composition containing, as its main components, a polymerizable monomer such as a (meth)acrylic polymerizable monomer, a polymerization initiator, and a filler is usually used.

In order to enhance the adhesion between the adhesive piece and the mouthpieces, it is preferable to use, as the adhesive piece, an adhesive piece that has been subjected to an end treatment such as formation of a lug portion so as to prevent the end portion from becoming loose. In addition, it is preferable that the adhesive piece has been subjected to a pretreatment such as impregnation with a thermosetting resin, a chemical polymerization resin, a photopolymerization resin, or the like.

The upper and lower mouthpieces 1 and 2 being “movable within a limited range” means that the upper and lower mouthpieces 1 and 2 to which the adhesive piece has been attached are movable in all directions, including, for example, upward, downward, forward, backward, leftward and rightward, relative to each other within a range of about 1 to 8 mm. That is, the upper and lower mouthpieces 1 and 2 are in an intermediate state in which they are not completely freely separated, or not completely restrained and fixed.

In this intermediate state, a patient with sleep apnea syndrome will not undergo obstruction of the air passage as a result of the tongue root portion drooping rearward during sleep. Moreover, the patient can move the lower jaw within a limited range even during sleep, and thus experiences a significantly reduced distress compared with when the mouthpieces are completely fixed.

In order to create an intermediate state with a proper movement distance according to the age, physique, symptom, and the like of a patient, it is preferable to appropriately adjust the raw material, the lengths in the vertical and transverse directions, the thickness, the weaving method, and the like of the adhesive piece 3.

### Embodiment 1

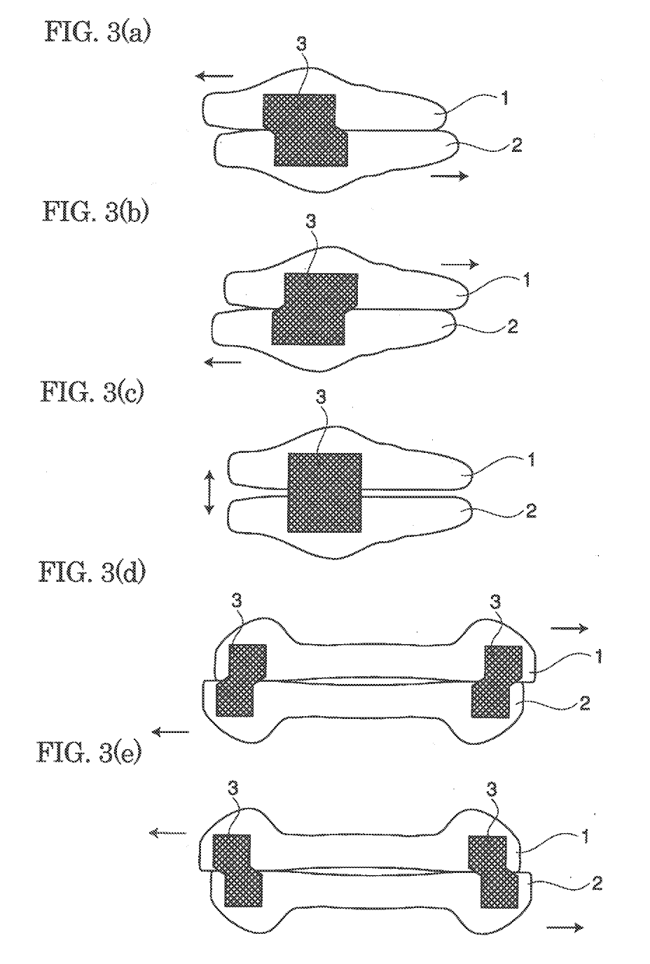

The upper and lower mouthpieces 1 and 2 shown in FIG. 1 have an adhesive piece 3 that is a 2.5×2.0 cm diagonal woven fabric made of glass fibers attached to either side. As shown in FIGS. 3(a) to 3(e), the upper and lower mouthpieces 1 and 2 were movable by about 1 mm in the up-down direction, and by about 5 mm in each of the leftward, upward, forward, and rearward directions.

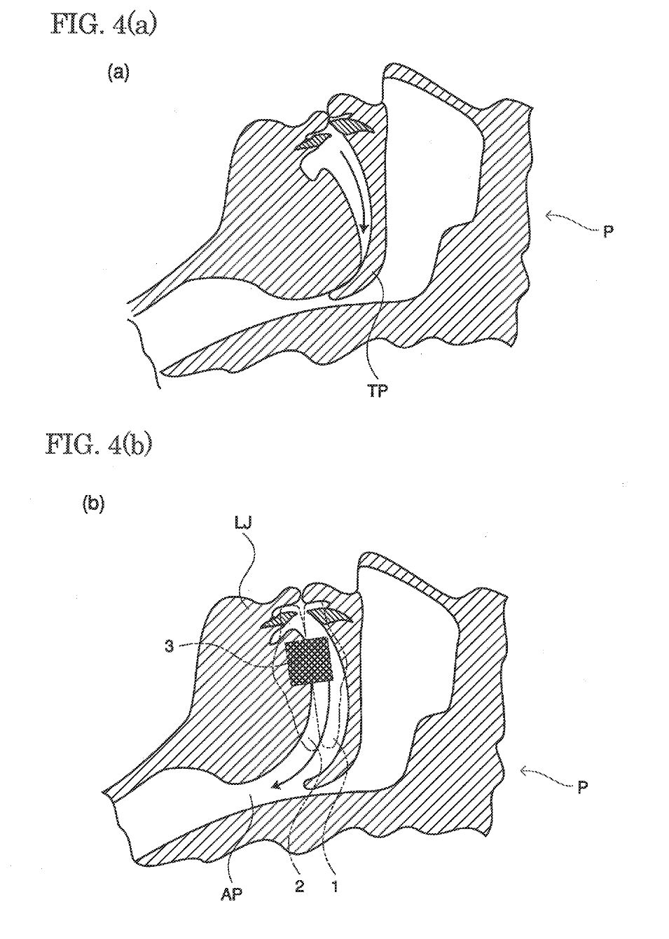

A patient P (male in his forties) with sleep apnea syndrome wore the upper and lower mouthpieces 1 and 2 during nighttime. Without the mouthpieces, the patient underwent obstruction of the air passage AP as a result of the tongue root portion TP drooping rearward as shown in FIG. 4(a). However, it was confirmed that, when the patient wore the mouthpieces 1 and 2, the air passage AP was expanded as shown in FIG. 4(b), and the obstructive sleep apnea and snoring were reduced.

Furthermore, it was confirmed that, when the patient wore the mouthpieces, the patient was able to move the lower jaw LJ by about 1 mm in the up-down direction, and by about 5 mm in each of the leftward, rightward, forward, and rearward directions even during sleep, and thus experiences a significantly reduced distress as compared with when the mouthpieces were completely fixed.

### Embodiments 2 to 6

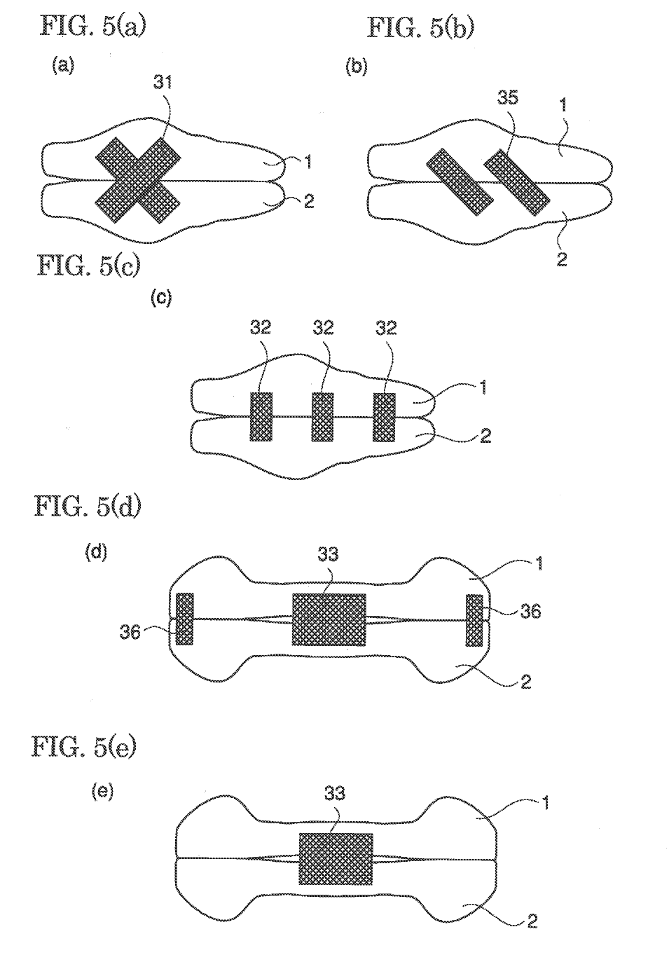

The upper and lower mouthpieces 1 and 2 shown in FIGS. 5(a) to 5(e) are five types of modifications of the shape, the number, and the attachment position of the adhesive piece. FIG. 5(a) is a modification using 1.5×2.0 cm adhesive pieces 31 disposed to form an X shape on either side. FIG. 5(d) is a modification using two 1.5×2.0 cm adhesive pieces 35 disposed diagonally parallel on either side. FIG. 5(c) is a modification using 1.5×2.0 cm adhesive pieces 32 respectively disposed at three locations on either side. FIG. 5(d) is a modification using a single 3.0×2.0 cm adhesive piece 33 disposed at the front and a 1.0×2.0 cm adhesive piece 36 disposed on either side. FIG. 5(e) is a modification using a single 3.0×2.0 cm adhesive piece 33 disposed only at the front. Various other configurations may be employed.

### Embodiment 7

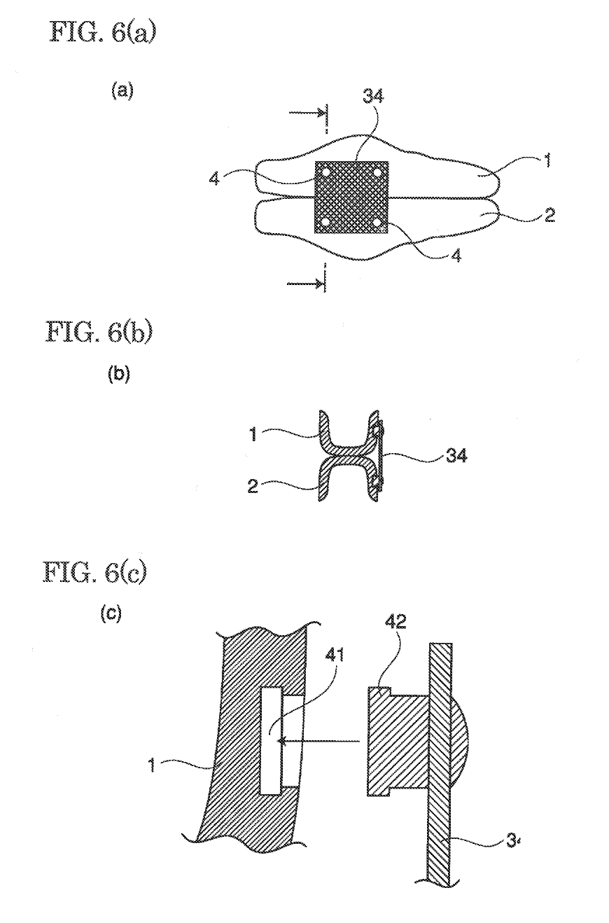

The upper and lower mouthpieces 1 and 2 shown in FIG. 6 are of a type that allows an adhesive piece 34 to be attached or removed with a one-touch snap button 4. Four pairs of the snap buttons 4 are disposed in FIG. 6(a). This type does not use an adhesive material.

As shown in FIGS. 6(a) to 6(c), female snap members 41 are formed integrally with the mouthpiece 1 on the mouthpieces 1 and 2 side, and male snap members 42 are attached to the adhesive piece 34 side. The attachment of the male snap members 42 to the adhesive piece 34 side can be performed by placing an adhesive piece 34 having a roughness that allows passage and penetration of molten resin by a molding pressure between upper and lower molds (not shown) defining a fastener molding space, and injecting a synthetic resin into the molding space. This is because the front surface side and the back surface side formed on opposite sides of the adhesive piece 34 are integrated with each other by the synthetic resin permeating the gaps between the stiches or weave patterns of the adhesive piece 34.

By removing the adhesive pieces 34 so as to allow the upper and lower mouthpieces to be freely moved, the mouthpieces can be widely used for usages other than that for sleep apnea syndrome. Examples of the usages include correction of the irregular teeth and the mandibular position, prevention of bruxism, and protection for sports that often involve contact with the face such as a combat sport. It is also possible to use only one of the upper and lower mouthpieces.

### REFERENCE SIGNS

1 Upper jaw-mouthpiece
2 Lower jaw-mouthpiece
3 Adhesive piece
31 to 36 Adhesive piece
4 Snap button
41 Female snap member
42 Male snap member
AP Air passage
LJ Lower jaw
P Patient
TP Tongue root portion

1. A therapeutic mouthpiece for sleep apnea syndrome and the like, comprising:
two separate upper jaw- and lower jaw-mouthpieces (1, 2), wherein
the upper jaw- and lower jaw-mouthpieces (1, 2) are bonded and fixed with at least one adhesive piece (3, 31 to 36), and the adhesive piece (3, 31 to 36) allows the upper jaw- and lower jaw-mouthpieces (1, 2) to be movable relative to each other within a limited range.

2. The mouthpiece according to claim 1, wherein
the limited range allows the upper jaw- and lower jaw-mouthpieces (1, 2) to move forward, rearward, leftward and rightward within a range of 1 to 8 mm.

3. The mouthpiece according to claim 1, wherein
a raw material of the adhesive piece (3, 31 to 36) is one of an inorganic fiber such as a glass fiber or a carbon fiber, and an organic fiber such as an aramid fiber or a polyethylene fiber.

4. The mouthpiece according to claim 1, wherein
the adhesive piece (3, 31 to 36) is a twilled fabric.

5. The mouthpiece according to claim 1, wherein
a quick cure resin is used as an adhesive material used for attaching the adhesive piece (3, 31 to 33, 35, 36) to the upper jaw- and lower jaw-mouthpieces (1, 2).

6. The mouthpiece according to claim 1, wherein
the adhesive piece (34) is attachable and removable to and from the upper jaw- and lower jaw-mouthpieces (1, 2).

7. The mouthpiece according to claim 6, wherein
a female or male snap member (41, 42) is disposed on the upper jaw- and lower jaw-mouthpieces (1, 2) and the adhesive piece (34) so as to allow the adhesive piece (34) to be attachable and removable to and from the upper jaw- and lower jaw-mouthpieces (1, 2).

8. An adhesive piece (3, 31 to 36) for use in bonding and fixing upper jaw- and lower jaw-mouthpieces (1, 2) so as to be movable relative to each other within a limited range.

9. The adhesive piece (3, 31 to 36) according to claim 8, wherein
the limited range allows the upper jaw- and lower jaw-mouthpieces (1, 2) to move at least forward, rearward, leftward, and rightward within a range of 1 to 8 mm.

10. The adhesive piece (3, 31 to 36) according to claim 8, wherein
a raw material of the adhesive piece is one of an inorganic fiber such as a glass fiber and a carbon fiber or an organic fiber such as an aramid fiber or a polyethylene fiber.

11. The adhesive piece (3, 31 to 36) according to claim 8, wherein
the adhesive piece is a twilled fabric.

12. The adhesive piece (3, 31 to 36) according to claim 8, wherein
the adhesive piece has been subjected to an end treatment such as formation of a lug portion so as to prevent end thereof from becoming loose.

13. The adhesive piece (3, 31 to 36) according to claim 8, wherein
the adhesive piece has been subjected to a pretreatment such as impregnation with a thermosetting resin, a chemical polymerization resin, a photopolymerization resin, or the like.
